(19) United States
(12) Patent Application Publication
(10) Pub. No.: US 2026/0014311 A1
(43) Pub. Date: Jan. 15, 2026

(54) APPARATUS, SYSTEMS AND METHODS FOR PURGING AN EXUDATE CANISTER

(71) Applicant: Solventum Intellectual Properties Company, Maplewood, MN (US)

(72) Inventors: Christopher J. CARROLL, San Antonio, TX (US); Javier GONZALEZ, San Antonio, TX (US); Jonathan G. REHBEIN, San Antonio, TX (US); Jordan TRAXLER, San Antonio, TX (US); Richard M. KAZALA, San Antonio, TX (US)

(21) Appl. No.: 18/992,036

(22) PCT Filed: Jul. 19, 2023
(86) PCT No.: PCT/IB2023/057353

§ 371 Date: Jan. 7, 2025

Related U.S. Application Data
(60) Provisional application No. 63/393,665, filed on Jul. 29, 2022.

(51) Int. Cl. A61M 1/00

(52) U.S. Cl. CPC A61M 1/98; A61M 1/631; A61M 1/912

(57) ABSTRACT

Apparatus, systems, and methods for purging exudate from a fluid container suitable for use with negative pressure therapy. A canister assembly configured to purge exudate can include a primary container, a first one-way valve, and a second one-way valve. The primary container can include an internal volume, a pressure inlet port, an exudate inlet port, and an exudate outlet port each in fluid communication with the internal volume. The first one-way valve can be in fluid communication with the exudate inlet port and the internal volume and configured to permit fluid flow into the internal volume and to restrict fluid flow out of the internal volume. The second one-way valve can be in fluid communication with the exudate outlet port and the internal volume and configured to permit fluid flow out of the internal volume and to restrict fluid flow into the internal volume.

### CROSS-REFERENCE TO RELATED APPLICATIONS

This application is a U.S. National Stage Entry of PCT International Application No. PCT/IB2023/057353, filed on Jul. 19, 2023, which claims the benefit of priority to U.S. Provisional Application No. 63/393,665, filed on Jul. 29, 2022, each of which are incorporated herein by reference in their entirety.

### TECHNICAL FIELD

The invention set forth in the appended claims relates generally to tissue treatment systems and more particularly, but without limitation, to apparatus, systems, and methods for purging an exudate canister.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatus, and methods for purging an exudate canister or reservoir in a negative-pressure therapy environment are set forth in the appended claims. Non-limiting illustrative example embodiments are also provided to enable a person skilled in the art to make and use the claimed subject matter.

For example, in some embodiments, a canister assembly configured to purge exudate can include a primary container, a first one-way valve, and a second one-way valve. The primary container can include an internal volume, a pressure inlet port, an exudate inlet port, and an exudate outlet port each in fluid communication with the internal volume. The first one-way valve can be in fluid communication with the exudate inlet port and the internal volume and configured to permit fluid flow into the internal volume and to restrict fluid flow out of the internal volume. The second one-way valve can be in fluid communication with the exudate outlet port and the internal volume and configured to permit fluid flow out of the internal volume and to restrict fluid flow into the internal volume.

Further, in some example embodiments, a system configured to purge exudate can include a primary container, a secondary container, a pump, and a valve. The primary container can include a first internal volume. The secondary container can include a second internal volume configured to be releaseably and fluidly coupled to the first internal volume. The pump can include a pump inlet and a pump outlet. The pump can be configured to generate negative pressure at the pump inlet and positive pressure at the pump outlet. The valve can be fluidly coupled to both the pump inlet and the pump outlet and selectively positionable in a therapy state or a purge state. The negative pressure at the pump inlet can be in fluid communication with the first internal volume in the therapy state, and the positive pressure at the pump outlet can be in fluid communication with the first internal volume in the purge state.

Further, in some example embodiments, a method of purging exudate from a canister can include providing a primary container including a first internal volume; providing a secondary container including a second internal volume; and discharging exudate from the first internal volume to the second internal volume.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a block diagram of an example embodiment of a therapy system that can provide negative-pressure treatment in accordance with this specification;

FIG. 2 is a cut-away view of an example therapy system deployed at an example tissue site;

FIG. 3 is a diagram of an example therapy system illustrating an example canister assembly and an example control valve positioned in a therapy state;

FIG. 4 is a diagram of the example therapy system and canister assembly of FIG. 3, illustrating the control valve positioned in a purge state;

FIG. 5A illustrates another example embodiment of a secondary container according to this specification;

FIG. 5B illustrates yet another example embodiment of a secondary container according to this specification;

FIG. 5C illustrates yet another example embodiment of a secondary container according to this specification; and

FIG. 6 illustrates yet another example embodiment of a secondary container according to this specification deployed with an example purge pump.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

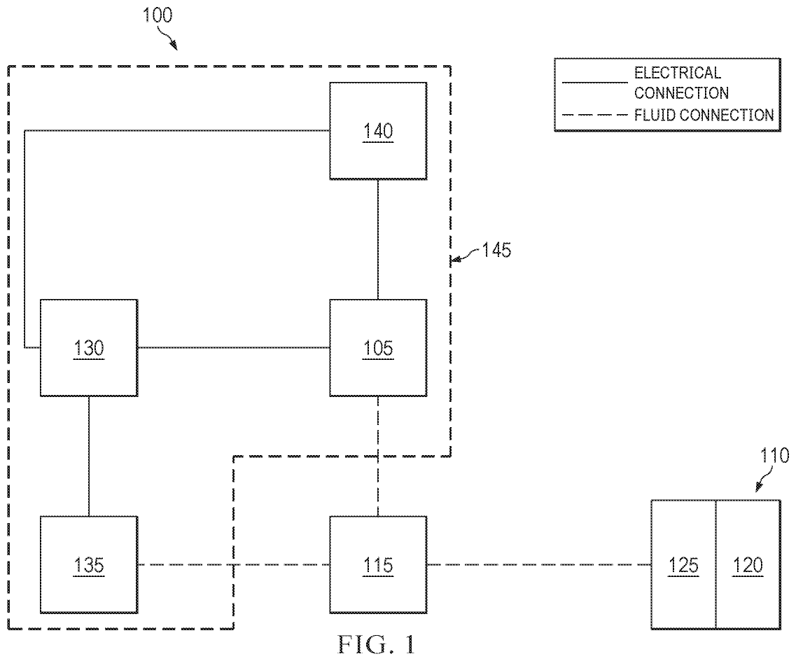

FIG. 1 is a block diagram of an example embodiment of a therapy system 100 that can provide negative-pressure therapy to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

Referring to FIG. 1, the therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a container 115, are examples of distribution components that may be associated with some examples of the therapy system 100. As illustrated in the example of FIG. 1, the dressing 110 may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from Kinetic Concepts, Inc. of San Antonio, Texas.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130 and other components into a therapy unit 145.

In general, components of the therapy system 100 may be coupled directly or indirectly. For example, the negative-pressure source 105 may be directly coupled to the container 115 and may be indirectly coupled to the dressing 110 through the container 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between-5 mm Hg (−667 Pa) and −500 mm Hg (−66.7 kPa). Common therapeutic ranges are between-50 mm Hg (−6.7 kPa) and −300 mm Hg (−39.9 kPa).

The container 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 130, may be a microprocessor or computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135 and the second sensor 140, may be any apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, in some embodiments. The signals from the first sensor 135 and the second sensor 140 may be suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise or consist essentially of a manifold. A manifold in this context may comprise or consist essentially of a means for collecting or distributing fluid relative to the tissue interface 120 under pressure. For example, a manifold may be adapted to receive negative pressure from a source and distribute negative pressure through multiple apertures across the tissue interface 120, which may have the effect of collecting fluid from a tissue site and drawing the fluid toward the source. In some embodiments, the fluid path may be reversed, or a secondary fluid path may be provided to facilitate delivering fluid to a tissue site.

In some illustrative embodiments, a manifold may comprise a plurality of pathways, which can be interconnected to improve distribution or collection of fluids. For example, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 120 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 120 may also vary according to needs of a prescribed therapy. For example, the tensile strength of foam may be increased for instillation of topical treatment solutions. The 25% compression load deflection of the tissue interface 120 may be at least 0.35 pounds per square inch, and the 65% compression load deflection may be at least 0.43 pounds per square inch. In some embodiments, the tensile strength of the tissue interface 120 may be at least 10 pounds per square inch. The tissue interface 120 may have a tear strength of at least 2.5 pounds per inch. In some embodiments, the tissue interface may be foam comprised of polyols such as polyester or polyether, isocyanate such as toluene diisocyanate, and polymerization modifiers such as amines and tin compounds. In some examples, the tissue interface 120 may be reticulated polyurethane foam such as found in GRANUFOAM™ dressing or V.A.C. VERAFLO™ dressing, both available from Kinetic Concepts, Inc. of San Antonio, Texas.

The thickness of the tissue interface 120 may also vary according to needs of a prescribed therapy. For example, the thickness of the tissue interface may be decreased to reduce tension on peripheral tissue. The thickness of the tissue interface 120 can also affect the conformability of the tissue interface 120. In some embodiments, a thickness in a range of about 5 millimeters to 10 millimeters may be suitable.

The tissue interface 120 may be either hydrophobic or hydrophilic. In an example in which the tissue interface 120 may be hydrophilic, the tissue interface 120 may also wick fluid away from a tissue site, while continuing to distribute negative pressure to the tissue site. The wicking properties of the tissue interface 120 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Texas. Other hydrophilic foams may include those made from polyether. Other foams that may exhibit hydrophilic characteristics include hydrophobic foams that have been treated or coated to provide hydrophilicity.

In some embodiments, the tissue interface 120 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 120 may further serve as a scaffold for new cell-growth, or a scaffold material may be used in conjunction with the tissue interface 120 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 125 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minnesota; polyurethane (PU) drape, commercially available from Avery Dennison Corporation, Pasadena, California; polyether block polyamide copolymer (PEBAX), for example, from Arkema S.A., Colombes, France; and Inspire 2301 and Inpsire 2327 polyurethane films, commercially available from Expopack Advanced Coatings, Wrexham, United Kingdom. In some embodiments, the cover 125 may comprise INSPIRE 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

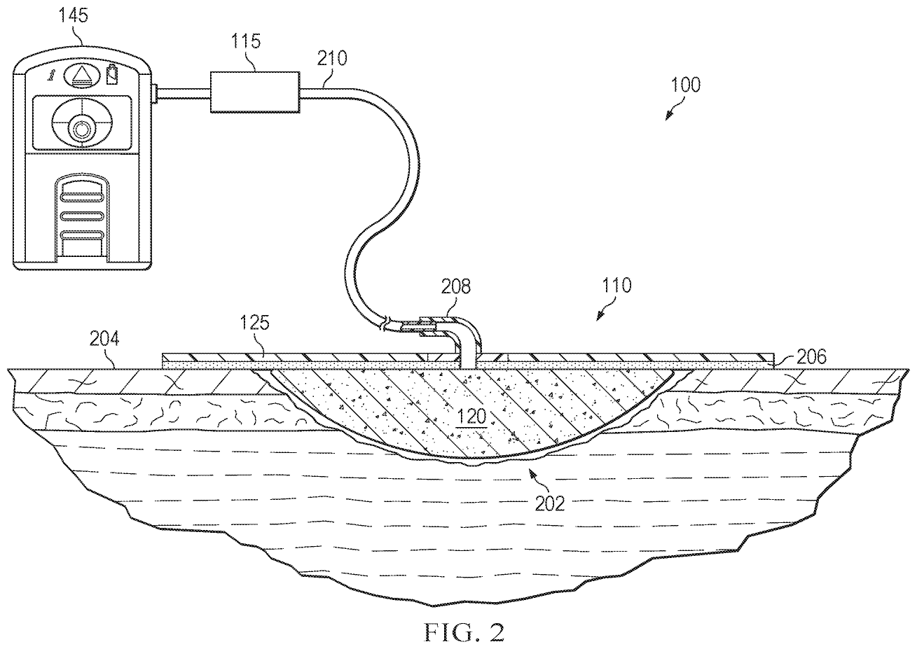

In operation, the tissue interface 120 may be placed within, over, on, or otherwise proximate to a tissue site, such as the tissue site 202 shown in FIG. 2. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis 204 peripheral to the tissue site 202. For example, an attachment device such as an adhesive layer 206 may be disposed around at least a perimeter of the cover 125 to secure the cover 125 to the epidermis 204. Thus, the dressing 110 can provide a sealed therapeutic environment proximate to a tissue site, substantially isolated from the external environment, and the negative-pressure source 105 can reduce pressure in the sealed therapeutic environment.

The process of reducing pressure may be described illustratively herein as “delivering,” “distributing,” or “generating” negative pressure, for example. In general, exudate and other fluid flow toward lower pressure along a fluid path. Thus, the term “downstream” typically implies a location in a fluid path relatively closer to a source of negative pressure or further away from a source of positive pressure. Conversely, the term “upstream” implies a location relatively further away from a source of negative pressure or closer to a source of positive pressure. However, the fluid path may also be reversed in some applications, such as by substituting a positive-pressure source for a negative-pressure source.

Negative pressure applied to a tissue site through the tissue interface 120 in a sealed therapeutic environment can induce macro-strain and micro-strain in the tissue site. Negative pressure can also remove exudate and other fluid from a tissue site, which can be collected in the container 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, the controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

FIG. 2 further illustrates an example of a dressing interface 208 fluidly coupling the dressing 110 to a fluid conductor 210. The dressing interface 208 may be, as one example, a port or connector, which permits the passage of fluid from the tissue interface 120 to the fluid conductor 210 and vice versa. In some embodiments, the dressing interface 208 may be an elbow connector. Fluid collected from the tissue site 202 may enter the fluid conductor 210 via the dressing interface 208. In other examples, the therapy system 100 may omit the dressing interface 208, and the fluid conductor 210 may be inserted directly through the cover 125 and into the tissue interface 120. In some examples, the fluid conductor 210 may have more than one lumen. For example, the fluid conductor 210 may have one lumen for negative pressure and liquid transport and one or more lumens for communicating pressure to a pressure sensor.

In operation, a negative pressure may be applied to the tissue interface 120 to cause fluid flow through the tissue interface 120 while the negative pressure is being delivered to the tissue interface 120. Fluid can move directly or indirectly toward the negative-pressure source 105 and into the container 115 through the tissue interface 120. Improved management of fluid or exudate captured in the container 115 can be beneficial to therapy systems. Accordingly, this specification further provides apparatus, systems, and methods for managing fluid or exudate captured in a container, such as the container 115. For example, as described further herein, exudate stored in a container, such as the container 115, can be purged from the container and captured in a secondary container that may be replaceable or reusable. This approach can expand the exudate storage capacity of a negative pressure therapy system to a larger capacity while maintaining a small form factor for the overall system.

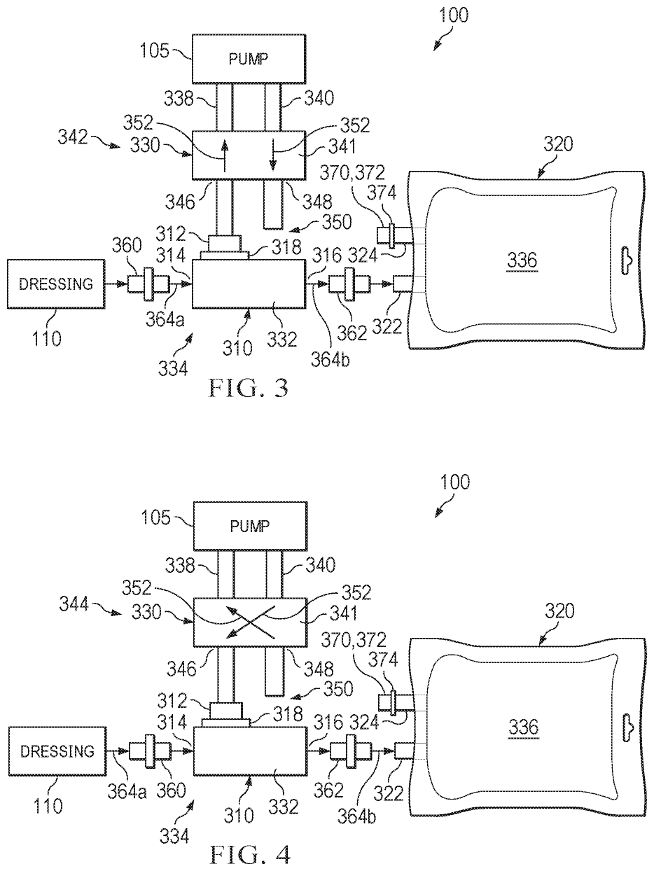

Referring to FIGS. 3-4, components of the therapy system 100, such as those associated with the therapy unit 145 illustrated in FIG. 1, may be omitted as desired or to suit the needs of a particular patient. FIGS. 3-4 illustrate another example embodiment of the therapy system 100 that can be configured to purge exudate. In some examples, the therapy system 100 can include a primary container 310, a secondary container 320, the pump 105, and a control valve 330.

The primary container 310 can be analogous to the container 115 introduced in FIG. 1 and can include a first internal volume 332 configured to collect fluid or exudate from a tissue site. Further, the primary container 310 can include a pressure inlet port 312, an exudate inlet port 314, and an exudate outlet port 316 each in fluid communication with the first internal volume 332. In some examples, a moisture barrier 318 can be positioned in fluid communication with the pressure inlet port 312 to prevent moisture or exudate from being communicated to the pump 105. The exudate inlet port 314 can be configured to be fluidly coupled to the dressing 110 and to receive exudate from a tissue site through the dressing 110. Further, the primary container 310 can be further configured as part of a canister assembly 334 configured to purge exudate into the secondary container 320 as described herein. In some examples, the purge of exudate may occur without interrupting the negative pressure therapy of the therapy system 100.

The secondary container 320 can include a second internal volume 336 configured to collect fluid or exudate from a tissue site. The secondary container 320 can be configured to be releasably and fluidly coupled to the first internal volume 332 of the primary container 310, such as, for example, with a suitable releasable fluid coupling. In some examples, the second internal volume 336 can be greater than the first internal volume 332. The second internal volume 336 of the secondary container 320 can be configured to be fluidly coupled to the first internal volume 332 of the primary container 310 through the exudate outlet port 316 of the primary container 310. For example, the secondary container 320 can include an inlet port 322 and an outlet port 324 each in fluid communication with the second internal volume 336. The inlet port 322 of the secondary container 320 can be configured to be fluidly coupled to the exudate outlet port 316 of the primary container 310. In some examples, the secondary container 320 can be removeable from the exudate outlet port 316 of the primary container 310.

In some examples, the primary container 310 can be formed of a rigid material and the secondary container 320 can be formed of a flexible material. In some examples, the secondary container 320 can be formed from one or more of a soft film, a nonwoven material, a rigid plastic, metal, and carbon fiber. In some examples, the primary container 310 can include walls formed of a rigid plastic, and the secondary container 320 can include walls formed of a soft film. For example, the secondary container 320 can be or can include a disposable bag.

The pump 105 can include a pump inlet 338 and a pump outlet 340. The pump 105 can be configured to generate negative pressure at the pump inlet 338 and positive pressure at the pump outlet 340. A valve body 341 of the control valve 330 can be fluidly coupled to both the pump inlet 338 and the pump outlet 340 and can be selectively positionable in a therapy state 342, shown in FIG. 3, or a purge state 344, shown in FIG. 4. The negative pressure at the pump inlet 338 can be in fluid communication with the first internal volume 332 of the primary container 310 and the dressing 110 in the therapy state 342. The positive pressure at the pump outlet 340 can be in fluid communication with the first internal volume 332 of the primary container 310 in the purge state 344 to force exudate from the first internal volume 332 into the second internal volume 336.

For example, the control valve 330 can include a first port 346 and a second port 348. The first port 346 can be configured to be in fluid communication with the primary container 310 through the pressure inlet port 312. The second port 348 can be in fluid communication with ambient environment 350. Flow passages 352 through the valve body 341 can be moved, switched, or re-oriented through the valve body 341 such that the control valve 330 can be configured to selectively switch the pump inlet 338 in fluid communication with the first port 346 or the second port 348, and the pump outlet 340 in fluid communication with the first port 346 or the second port 348, depending on the state of therapy or whether a user desires to purge exudate from the primary container 310. For example, the negative pressure at the pump inlet 338 can be in fluid communication with the first port 346, and the positive pressure or exhaust from the pump outlet 340 can be in fluid communication with the second port 348 in the therapy state 342. Conversely, the negative pressure at the pump inlet 338 can be in fluid communication with the second port 348, and the positive pressure or exhaust at the pump outlet 340 can be in fluid communication with the first port 346 in the purge state 344. In some examples, the control valve 330 can be or can include a 4/2 directional solenoid control valve, such as a Direct Operated Balanced 4-Way Valve, available from Humphreys, USA. As an alternative to a 4/2 directional solenoid control valve, a combination of two 3/2 directional solenoid control valves can be used as the control valve 330. A suitable 3/2 directional solenoid control valve can be a 3/2 Parker X-Valve®, available from Parker Hannifin of Hollis, New Hampshire, USA.

The canister assembly 334 can include the primary container 310, a first one-way valve 360, and a second one-way valve 362. The first one-way valve 360 can be in fluid communication with the exudate inlet port 314 and the first internal volume 332 and be configured to permit fluid flow into the first internal volume 332 and to restrict fluid flow out of the first internal volume 332 so that fluid or exudate cannot return to the dressing 110 from the primary container 310, for example, during the purge state 344. The second one-way valve 362 can be in fluid communication with the exudate outlet port 316 and the first internal volume 332 and be configured to permit fluid flow out of the first internal first volume 332 and to restrict fluid flow into the first internal volume 332 so that ambient air or fluid from the second internal volume 336 cannot be drawn into the first internal volume 332, for example, during the therapy state 342. As described, the first one-way valve 360 and the second one-way valve 362 each include a flow direction 364 positioned opposite each other relative to the primary container 310. For example, the first one-way valve 360 can have a first flow direction 364a directed toward or into the first internal volume 332, and the second one-way valve 362 can have an opposite second flow direction 364b directed away from or out of the internal volume 332. In some examples, one or both of the first one-way valve 360 and the second one-way valve 362 can be or can include a check valve, flap valve, duck-bill valve, or a solenoid valve configured direct fluid flow in the first flow direction 364a and the second flow direction 364b as described.

In some examples, additionally or alternatively to the second one-way valve 362, a removeable and replaceable cap (not shown), such as a threaded cap, can be used to seal the exudate outlet port 316 during the therapy state 342. The cap can be removed when a user desires to purge the primary container 310 in the purge state 344.

Additional valves can be deployed to control and manage fluid flow through the therapy system 100. For example, the outlet port 324 of the secondary container 320 can be in fluid communication with a third one-way valve 370 configured to permit fluid flow out of or away from the second internal volume 336 and to prevent fluid flow into or toward the second internal volume 336. In some examples, the outlet port 324 of the secondary container 320 can be or can include a vent 372 in fluid communication with ambient environment. Further, in some examples, a moisture barrier 374 can be positioned in fluid communication with the outlet port 324 of the secondary container 320. The moisture barrier 374 can prevent liquid and exudates from leaving the second internal volume 336, but permit gases to escape.

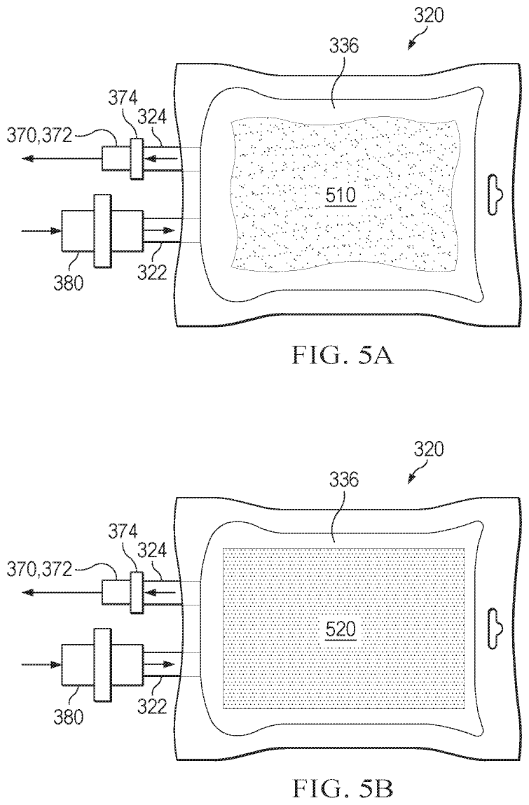

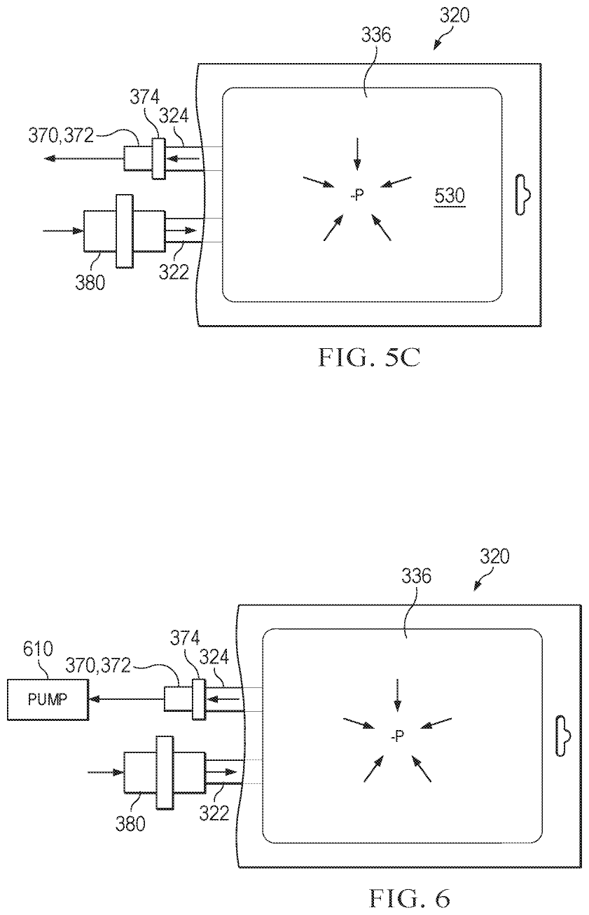

Referring to FIGS. 5A-6, in some examples, a fourth one-way valve 380 can be positioned in fluid communication with the inlet port 322 of the secondary container 320 and the second internal volume 336. The fourth one-way valve 380 can be configured to permit fluid flow into the second internal volume 336 and to restrict fluid flow out of the second internal volume 336 analogous to the second one-way valve 362 and as shown by the directional arrows. The fourth one-way valve 380 can be coupled inline with the second one-way valve 362 by, for example, any suitable fluid coupling or releasable coupling (not shown) positioned between the second one-way valve 362 and the fourth one-way valve 380. The fourth one-way valve 380 may prevent or assist in the prevention of spillage of exudate from the second internal volume 336 when or if the secondary container 320 is disconnected from the primary container 310 between the second one-way valve 362 and the fourth one-way valve 380, for example, at the fluid coupling.

Referring to FIG. 5A, in some examples, the secondary container 320 can include a fluid solidifier 510 positioned in the second internal volume 336.

Referring to FIG. 5B, in some examples, the secondary container 320 can include a fluid receptor 520, which can be or can include one or more of a wicking material, a superabsorbent material, and high suction capillary foam positioned in the second internal volume 336. The fluid receptor 520 can enhance flow into the secondary container with or without the need for forced flow from a pump, for example. Further, the fluid receptor 520 can maintain an open space and help prevent collapse of the second internal volume 336.

Referring to FIG. 5C, in some examples, the secondary container 320 can include a captive negative pressure 530 contained within the second internal volume 336 that is configured to be communicated to the first internal volume 332 when the inlet port 322 of the secondary container 320 is fluidly coupled to the exudate outlet port 316 of the primary container 310. For example, the captive negative pressure 530 can be generated inside the second internal volume 336 from an external source or supply of negative pressure prior to the secondary container 320 being fluidly coupled to the exudate outlet port 316. Additionally or alternatively, the captive negative pressure 530 can be generated by a compressed foam, such as a high suction capillary foam, positioned within the second internal volume 336. The compressed foam can be permitted to expand to a greater, uncompressed volume when the secondary container 320 is fluidly coupled to the exudate outlet port 316 of the primary container 310. The expansion in volume of the compressed foam from a compressed state to an uncompressed state can generate negative pressure within the second internal volume 336, which can be communicated to the first internal volume 332 to draw exudate from the first internal volume 332.

Referring to FIG. 6, in some examples, additionally or alternatively to the pump 105, a purge pump 610 can be provided as source of negative and/or positive pressure that can be deployed to cause or enhance the purge of exudate from the primary container 310 to the secondary container 320. As shown in FIG. 6, the purge pump 610 can be configured to be fluidly coupled to the outlet port 324 of the secondary container 320. In such a configuration, the purge pump 610 can be configured to provide negative pressure to the outlet port 324 of the secondary container 320, which will be communicated to the primary container 310 through the second internal volume 336 and the connection of the inlet port 322 of the secondary container 320 to the exudate outlet port 316 of the primary container 310. In another example embodiment (not shown), the purge pump 610 can, by way of analogy, be configured to provide positive pressure to the pressure inlet port 312 of the primary container 310 to force exudate from the first internal volume 332 to the second internal volume 336.

Further provided are methods for purging exudate from a canister, such as the primary container 310. For example, in some embodiments, a method of purging exudate from a canister can include providing the primary container 310 including the first internal volume 332; providing the secondary container 320 including the second internal volume 336; and discharging exudate from the first internal volume 332 to the second internal volume 336.

In some examples, discharging exudate from the first internal volume 332 to the second internal volume 336 can include fluidly coupling the first internal volume 332 to the second internal volume 336 and applying positive pressure to the first internal volume 332 through the pressure inlet port 312 on the primary container 310 to force the exudate through the exudate outlet port 316 and into the second internal volume 336.

Additionally or alternatively, in some examples, discharging exudate from the first internal volume 332 to the second internal volume 336 can include fluidly coupling the first internal volume 332 to the second internal volume 336 and wicking the exudate from the first internal volume 332 to the second internal volume 336 by operation of one or more of a wicking material, a superabsorbent material, or a high suction capillary foam being positioned in the second internal volume 336 and exposed to the first internal volume 332.

Additionally or alternatively, in some examples, discharging exudate from the first internal volume 332 to the second internal volume 336 can include fluidly coupling the first internal volume 332 to the second internal volume 336 and applying negative pressure to the first internal volume 332 through the second internal volume 336. For example, negative pressure can be applied to the second internal volume 336 through the outlet port 324 of the secondary container 320. The negative pressure in the second internal volume 336 can be communicated through the inlet port 322 of the secondary container 320 and through the exudate outlet port 316 of the primary container 310 to the first internal volume 332, which will draw exudate from the first internal volume 332 into the second internal volume 336.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use. For example, in some configurations the dressing 110, the container 115, or both may be eliminated or separated from other components for manufacture or sale. In other example configurations, the controller 130 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A canister assembly configured to purge exudate, comprising:
a primary container including an internal volume, a pressure inlet port, an exudate inlet port, and an exudate outlet port each in fluid communication with the internal volume;
a first one-way valve in fluid communication with the exudate inlet port and the internal volume, the first one-way valve configured to permit fluid flow into the internal volume and to restrict fluid flow out of the internal volume; and
a second one-way valve in fluid communication with the exudate outlet port and the internal volume, the second one-way valve configured to permit fluid flow out of the internal volume and to restrict fluid flow into the internal volume.

2. (canceled)

3. The canister assembly of claim 1, wherein the first one-way valve and the second one-way valve each include a flow direction positioned opposite each other relative to the primary container.

4. (canceled)

5. The canister assembly of claim 1, wherein the exudate inlet port is configured to be fluidly coupled to a dressing positioned at a tissue site and to receive exudate from the tissue site.

6. The canister assembly of claim 1, wherein the internal volume of the primary container is a first internal volume, and wherein the canister assembly further comprises a secondary container including a second internal volume that is configured to be fluidly coupled to the first internal volume through the exudate outlet port of the primary container.

7. The canister assembly of claim 6, wherein the secondary container includes an inlet port and an outlet port each in fluid communication with the second internal volume, the inlet port of the secondary container configured to be fluidly coupled to the exudate outlet port of the primary container, and the outlet port of the secondary container in fluid communication with a third one-way valve configured to permit fluid flow out of the second internal volume and to prevent fluid flow into the second internal volume.

8. The canister assembly of claim 7, further comprising a moisture barrier positioned in fluid communication with the outlet port of the secondary container.

9. (canceled)

10. The canister assembly of claim 7, further comprising a fourth one-way valve in fluid communication with the inlet port of the second container and the second internal volume, the fourth one-way valve configured to permit fluid flow into the second internal volume and to restrict fluid flow out of the second internal volume.

11. The canister assembly of claim 6, wherein the secondary container is removeable from the exudate outlet port of the primary container.

12. The canister assembly of claim 6, wherein the second internal volume is greater than the first internal volume.

13. The canister assembly of claim 6, wherein the primary container is formed of a rigid material and the secondary container is formed of a flexible material.

14. (canceled)

15. The canister assembly of claim 6, wherein the primary container comprises walls formed of a rigid plastic and the secondary container comprises walls formed of a soft film.

16. The canister assembly of claim 6, wherein the secondary container comprises a disposable bag.

17. The canister assembly of claim 6, wherein the secondary container comprises a fluid solidifier positioned in the second internal volume.

18. The canister assembly of claim 6, wherein the secondary container includes one or more of a wicking material, a superabsorbent material, and high suction capillary foam positioned in the second internal volume.

19. The canister assembly of claim 10, wherein the secondary container includes a captive negative pressure within the second internal volume that is configured to be communicated to the first internal volume when the inlet port of the secondary container is fluidly coupled to the exudate outlet port of the primary container.

20. The canister assembly of claim 7, further comprising a purge pump configured to be fluidly coupled to the pressure inlet port of the primary container or the outlet port of the secondary container, wherein the purge pump is configured to provide positive pressure to the pressure inlet port of the primary container or negative pressure to the outlet port of the secondary container.

21. The canister assembly of claim 1, further comprising a pump including a pump inlet and a pump outlet, the pump configured to generate negative pressure at the pump inlet and positive pressure at the pump outlet.

22. The canister assembly of claim 21, further comprising a control valve fluidly coupled to both the pump inlet and the pump outlet and including a first port and a second port, first port configured to be in fluid communication with the primary container through the pressure inlet port, the second port configured to be in fluid communication with ambient environment.

23. The canister assembly of claim 22, wherein the control valve is selectively positionable in a therapy state or a purge state, wherein the negative pressure at the pump inlet is in fluid communication with the first port and the pump outlet is in fluid communication with the second port in the therapy state, and wherein the pump inlet is in fluid communication with the second port and the positive pressure at the pump outlet is in fluid communication with the first port in the purge state.

24. (canceled)

25. A system configured to purge exudate, comprising:
a primary container including a first internal volume;
a secondary container including a second internal volume configured to be releaseably and fluidly coupled to the first internal volume;
a pump including a pump inlet and a pump outlet, the pump configured to generate negative pressure at the pump inlet and positive pressure at the pump outlet;
a control valve fluidly coupled to both the pump inlet and the pump outlet and being selectively positionable in a therapy state or a purge state, wherein the negative pressure at the pump inlet is in fluid communication with the first internal volume in the therapy state, and wherein the positive pressure at the pump outlet is in fluid communication with the first internal volume in the purge state.

26. A method of purging exudate from a canister, comprising:
providing a primary container including a first internal volume;
providing a secondary container including a second internal volume; and
discharging exudate from the first internal volume to the second internal volume.

27.-30. (canceled)
